(19) United States
(12) Patent Application Publication
(10) Pub. No.: US 2025/0037873 A1
(43) Pub. Date: Jan. 30, 2025

(54) STRESS ESTIMATION METHOD

(71) Applicant: NEC Corporation, Minato-ku, Tokyo (JP)

(72) Inventors: Tasuku KITADE, Tokyo (JP); Masanori TSUJIKAWA, Tokyo (JP); Keisuke SUZUKI, Tokyo (JP)

(73) Assignee: NEC Corporation, Minato-ku, Tokyo (JP)

(21) Appl. No.: 18/713,777

(22) PCT Filed: Dec. 10, 2021
(86) PCT No.: PCT/JP2021/045653

§ 371 Date: May 28, 2024

(51) Int. Cl. G16H 50/30

(52) U.S. Cl. CPC G16H 50/30

(57) ABSTRACT

A stress estimating device 100 of the present invention includes: a first calculating unit 121 that calculates a first stress value by inputting, to a first stress calculation model, first data based on biometric data obtained by measurement from a target person; and a second calculating unit 122 that calculates a second stress value by inputting, to a second stress calculation model, the first stress value, and second data based on stress-related subjective data acquired from the target person, in which the second stress calculation model is generated in advance by learning using the first stress value and the second data about a predetermined person acquired in the past.

### TECHNICAL FIELD

The present invention relates to a stress estimation method, a stress estimating device, and a program.

### BACKGROUND ART

As methods of estimating stress on a person, methods based on the subjectivity of a person, and methods based on biometric information about a person are known. In the method based on the subjectivity of a person, for example, stress is estimated on the basis of answers from the person to a predetermined questionnaire. In addition, in the method based on biometric information about a person, stress is estimated on the basis of the biometric information about the person acquired from a wearable terminal or images.

Here, in Patent Literature 1, final stress is estimated on the basis of a stress value based on the subjectivity of a person, and a stress value based on biometric information about the person. Specifically, in Patent Literature 1, a coordinate plane defined by a coordinate axis related to a subjective stress value, and a coordinate axis related to an objective stress value is prepared in advance, and the final stress is estimated on the basis of an area on the coordinate plane where an acquired subjective stress value and objective stress value are positioned. In addition, in Patent Literature 1, stress is estimated also by calculating a total stress value using the subjective stress value and the objective stress value, and a predefined function.

### CITATION LIST

### Patent Literature

Patent Literature 1: JP 2017-169974 A

### SUMMARY OF INVENTION

### Technical Problem

However, in the method in Patent Literature 1 mentioned above, since a final stress-related state is determined by preset criteria (the coordinate plane and the function) on the basis of the subjective stress value and the objective stress value, and the determination criteria are applied uniformly, it is difficult to estimate stress more precisely.

Therefore, an object of the present invention is to provide a stress estimation method that can solve the problem mentioned above that stress cannot be estimated more highly precisely.

### Solution to Problem

A stress estimation method according to one aspect of the present invention includes:
calculating a first stress value by inputting, to a first stress calculation model, first data based on biometric data obtained by measurement from a target person; and
calculating a second stress value by inputting, to a second stress calculation model, the first stress value, and second data based on stress-related subjective data acquired from the target person, in which
the second stress calculation model is generated in advance by learning using the first stress value and the second data about a predetermined person acquired in a past.

In addition, a stress estimating device according to one aspect of the present invention includes:
a first calculating unit that calculates a first stress value by inputting, to a first stress calculation model, first data based on biometric data obtained by measurement from a target person; and
a second calculating unit that calculates a second stress value by inputting, to a second stress calculation model, the first stress value, and second data based on stress-related subjective data acquired from the target person, in which
the second stress calculation model is generated in advance by learning using the first stress value and the second data about a predetermined person acquired in a past.

In addition, a program according to one aspect of the present invention causes an information processing device to execute processes of:
calculating a first stress value by inputting, to a first stress calculation model, first data based on biometric data obtained by measurement from a target person; and
calculating a second stress value by inputting, to a second stress calculation model, the first stress value, and second data based on stress-related subjective data acquired from the target person, in which
the second stress calculation model is generated in advance by learning using the first stress value and the second data about a predetermined person acquired in a past.

### Advantageous Effects of Invention

By being configured in the manners described above, the present invention makes it possible to estimate stress more highly precisely.

### BRIEF DESCRIPTION OF DRAWINGS

FIG. 1 is a block diagram depicting the configuration of a stress estimating device in a first exemplary embodiment of the present invention.

FIG. 2 is a figure depicting an example of a test for acquiring stress-related subjective data about people input to the stress estimating device disclosed in FIG. 1.

FIG. 3 is a flowchart depicting an operation performed by the stress estimating device disclosed in FIG. 1.

FIG. 4 is a flowchart depicting an operation performed by the stress estimating device disclosed in FIG. 1.

FIG. 5 is a figure for explaining advantageous effects achieved with the stress estimating device in the first exemplary embodiment of the present invention.

FIG. 6 is a block diagram depicting the hardware configuration of a stress estimating device in a second exemplary embodiment of the present invention.

FIG. 7 is a block diagram depicting the configuration of the stress estimating device in the second exemplary embodiment of the present invention.

FIG. 8 is a flowchart depicting an operation performed by the stress estimating device in the second exemplary embodiment of the present invention.

### DESCRIPTION OF EMBODIMENTS

### First Exemplary Embodiment

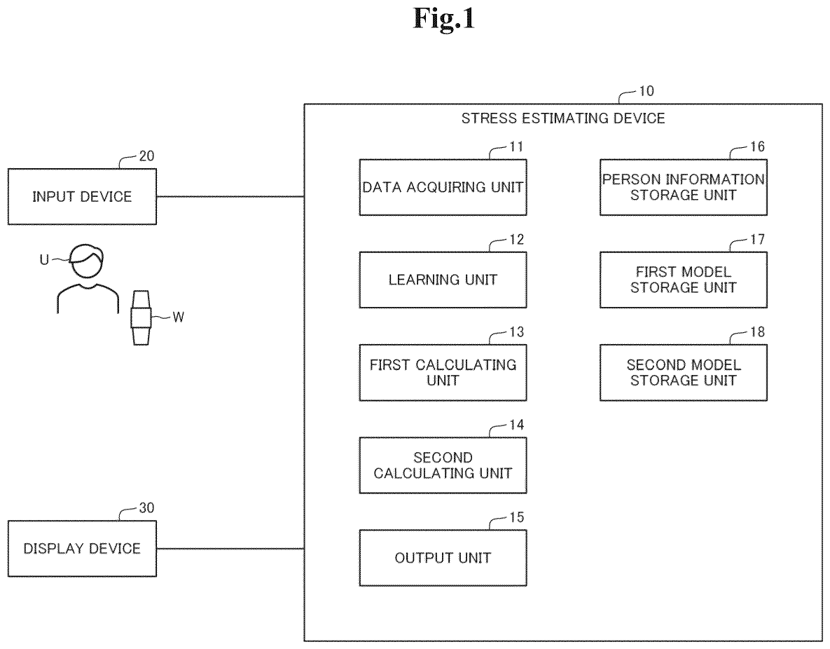

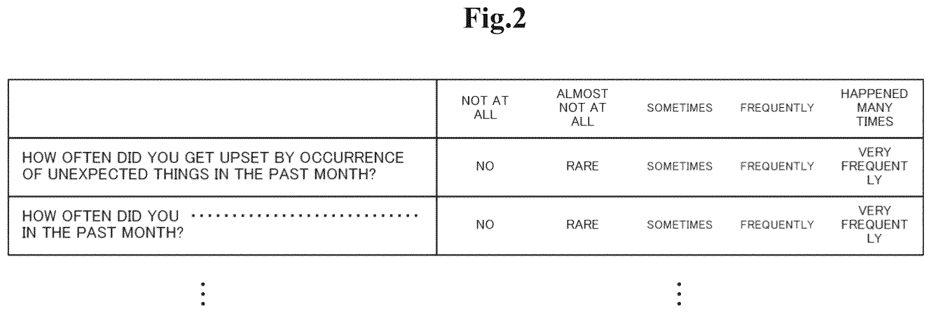

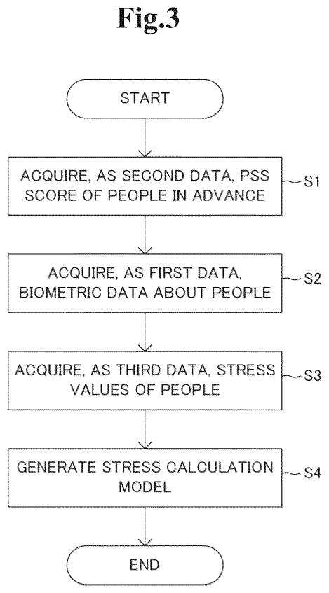

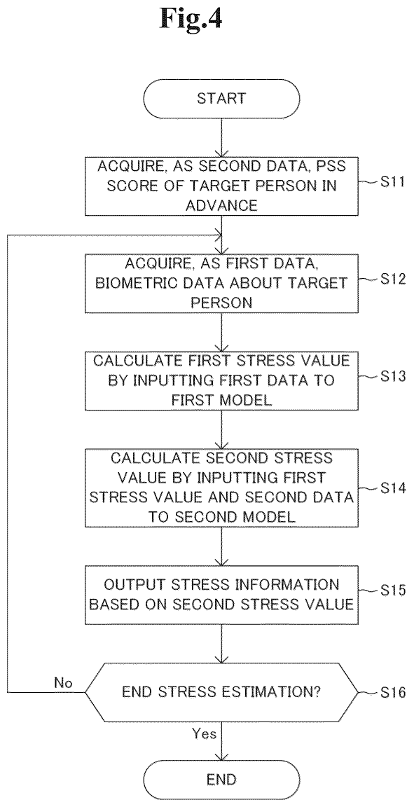

A first exemplary embodiment of the present invention is explained with reference to FIG. 1 to FIG. 4. FIG. 1 to FIG. 2 are figures for explaining the configuration of a stress estimating device, and FIG. 3 to FIG. 4 are figures for explaining processing operations performed by the stress estimating device.

### Configuration

A stress estimating device 10 in the present invention is used for estimating stress on a person. For example, the stress estimating device 10 is used for calculating a stress value representing a chronic or acute stress-related state of a person. It should be noted that the stress estimating device 10 in the present invention may calculate any stress value of a person.

The stress estimating device 10 is configured using one or more information processing devices including an arithmetic device and a storage device. Then, as depicted in FIG. 1, the stress estimating device 10 includes a data acquiring unit 11, a learning unit 12, a first calculating unit 13, a second calculating unit 14, and an output unit 15. Respective functions of the data acquiring unit 11, the learning unit 12, the first calculating unit 13, the second calculating unit 14, and the output unit 15 can be realized by the arithmetic device executing programs that are stored on the storage device, and are for realizing the respective functions. In addition, the stress estimating device 10 includes a person information storage unit 16, a first model storage unit 17, and a second model storage unit 18. The person information storage unit 16, the first model storage unit 17, and the second model storage unit 18 are configured using the storage device. Hereinbelow, the respective configurations are mentioned in detail.

The data acquiring unit 11 (acquiring unit) acquires data to be used for estimating stress on a person. First, the data acquiring unit 11 acquires learning data for generating, by machine learning, a stress calculation model to be used for calculating a stress value. The learning data is data about certain many people (predetermined people), and is stored on the person information storage unit 16 in association with each person.

Specifically, the data acquiring unit 11 acquires, as learning data, second data which is data based on stress-related subjective data about the people. At this time, as the second data which is data based on stress-related subjective data about the people, the data acquiring unit 11 acquires “PSS scores” obtained by totaling “perceived stress scales (Perceived Stress Scales (PSSs)).” Here, PSSs include 14 preset question items for asking a user about how she/he is feeling about things happening to her/him as depicted in FIG. 2 as an example, and five levels of answers are prepared. The five levels of answers are given scores from 0 to 4, and the total of the scores of answers to 10 computation-target question items in the 14 questions is calculated as a PSS score. Therefore, the value of the PSS score ranges from 0 to 40. As an example, the data acquiring unit 11 causes an input device 20 like an information processing terminal operated by a person U as depicted in FIG. 1 to display PSS questions like the ones depicted in FIG. 2, acquires answers input by the person U to the input device 20, and acquires a PSS score by totaling the answers. It should be noted that the data acquiring unit 11 does not necessarily acquire data represented by one numerical value as the data based on stress-related subjective data about the people, but may acquire data represented by a plurality of numerical values. In addition, the data acquiring unit 11 does not necessarily acquire data represented by numerical values, but may acquire data represented by any values.

In addition, as learning data, the data acquiring unit 11 acquires first data which is data based on biometric data about the people obtained from the people in everyday life or during duties. For example, the data acquiring unit 11 acquires, as the biometric data, a heart rate of a person U via a measurement device such as a wearable terminal W worn by the person U as depicted in FIG. 1, or acquires, as the biometric data, an eye opening degree extracted from a facial image of the person U captured with a camera which is not depicted. Note that the data acquiring unit 11 may acquire, as learning data, the biometric data itself obtained by measurement or the first data including feature values extracted from the biometric data obtained by measurement. It should be noted that the data acquiring unit 11 may acquire any biometric data as the biometric data about the people using any measurement device.

In addition, as learning data, the data acquiring unit 11 acquires third data which is data representing the stress-related condition of the people U at the time of measurement for the biometric data about the people as the first data as mentioned above. For example, the data acquiring unit 11 asks a person U preset questions via the input device 20 during acquisition of the biometric data or in a predetermined period before or after the acquisition, acquires and totals answers from the person U, and thereby acquires the data based on stress-related subjective data about the person U. At this time, the data acquired as the third data is data calculated from the answers to a plurality of the questions for asking the degree of mental burden on the person U around the timing of measurement for the biometric data. As an example, in a case where a PSS score mentioned above is used as the third data, the third data represents a stress-related state during approximately one month around the time of measurement for the biometric data about the person. It should be noted that the third data may be data that is based on answers to another stress test, and represents stress-related states during measurement for the biometric data about the people or stress-related states in a predetermined period including the time of measurement for the biometric data.

The learning unit 12 performs machine learning using the learning data acquired as mentioned above, and generates a stress calculation model for calculating a stress value representing the stress-related condition of a person U. Specifically, before the machine learning, first, the learning unit 12 calculates first stress values from the biometric data itself about the people U obtained by measurement as mentioned above or the first data which is feature values of the biometric data. At this time, the learning unit 12 inputs the first data to a first stress calculation model stored on the first model storage unit 17, and thereby calculates the first stress values which are outputs therefrom. Note that the first stress calculation model is configured as a model that is preset to calculate a stress value corresponding to biometric data about a person U. For example, the first stress calculation model may be: a model generated by machine learning of biometric data and stress-related subjective data acquired from people until then; a model expressed by an arithmetic expression generated by manually examining biometric data, subjective data, and the like; furthermore a model expressed by an arithmetic expression generated from biometric data in accordance with a predetermined theory; or the like. As an example, the first stress calculation model is a model configured to calculate a value from 0 to 40 as an output similarly to a PSS score which is the second data mentioned above, using, as an input, the first data which is biometric data about a person U. It should be noted that the first stress calculation model may be a model that calculates a value in any format as an output.

Next, the learning unit 12 generates a second stress calculation model that calculates a second stress value by performing learning using the first stress values calculated using the first stress calculation model from the first data based on biometric data about the people U as mentioned above, and the second data which is stress-related subjective data like PSS scores acquired in advance from the people U. At this time, the learning unit 12 generates the second stress calculation model by performing machine learning using, as explanatory variables, the first stress values and second data described above, and using, as a response variable, the third data representing the stress-related condition of the people U at the time of acquisition of the biometric data from which the first stress values have been calculated. Then, the learning unit 12 stores the generated second stress calculation model on the second model storage unit 18.

Next, functions of respective units at the time of estimation of stress on a target person (target person) using the second stress calculation model after the second stress calculation model is generated as mentioned above are explained. For example, it is assumed, as a scene, that a target person U always keeps measuring biometric data with a wearable terminal or the like as mentioned later, and estimates a chronic stress value once a day. It should be noted that the scene also may be a scene where the person U estimates stress at any frequency such as a scene where the person U estimates an acute stress value every hour. Note that whereas the target person U is a person different from any of the certain many people having provided the learning data as mentioned above, the target person U may be any of the certain people having provided the learning data.

The data acquiring unit 11 (acquiring unit) acquires data to be used for estimating stress on the target person U. Specifically, as the second data which is data based on stress-related subjective data about the target person U, first, the data acquiring unit 11 acquires a “PSS score” obtained by totaling “perceived stress scales (Perceived Stress Scales (PSSs))” in advance. The PSS score to be acquired as the second data at this time is data acquired similarly to the learning data mentioned above, and, for example, is a value from 1 to 40 calculated on the basis of answers input by the person U via the input device 20 to questions, an example of which is depicted in FIG. 2, as mentioned above. Then, the data acquiring unit 11 stores, on the person information storage unit 16, the acquired PSS score as the second data about the target person U. Note that the second data about the target person U is regularly acquired and updated, for example, when the person U is registered in a system that performs stress estimation when she/he starts belonging to a workplace, and so on, once a year, and so on. It should be noted that the data acquiring unit 11 does not necessarily acquire a PSS score represented by one value as mentioned above as the data used for estimating stress, but may acquire data represented by a plurality of numerical values. In addition, the data acquiring unit 11 does not necessarily acquire data represented by numerical values, but may acquire data represented by any values.

In addition, the data acquiring unit 11 acquires the first data which is data based on biometric data from the target person U when stress on the person U is actually estimated after the second data like a PSS score is acquired in advance from the target person U as mentioned above. For example, the data acquiring unit 11 acquires, as the biometric data, a heart rate of the person U that is always kept being obtained by measurement at a measurement device such as the wearable terminal W worn by the person U as depicted in FIG. 1, or acquires, as the biometric data, an eye opening degree extracted from a facial image of the person U captured with a camera which is not depicted. Then, the data acquiring unit 11 acquires the first data based on biometric data from the person U at preset timings (e.g. at certain time intervals) or at any timings (irregularly). As an example, the data acquiring unit 11 acquires the first data every day in a case where chronic stress on the target person U is estimated, or every hour in a case where acute stress is measured. Note that the data acquiring unit 11 may acquire, as the first data, the biometric data itself obtained by measurement, or may acquire, as the first data, feature values extracted, by a preset method, from the biometric data obtained by measurement. It should be noted that the data acquiring unit 11 may acquire any biometric data as the biometric data about the person using any measurement device.

Every time the first data based on biometric data is acquired from the person U as mentioned above, the first calculating unit 13 calculates the first stress value from the first data. At this time, the first calculating unit 13 reads out the first stress calculation model stored on the first model storage unit 17, inputs the acquired first data to the first stress calculation model, and thereby calculates the first stress value which is an output therefrom. That is, at preset timings such as at certain time intervals, the data acquiring unit 11 and the first calculating unit 13 cooperate with each other to repeat the acquisition of the first data based on biometric data from the person U, and the calculation of the first stress value based on the biometric data every time the first data is acquired. For example, the first calculating unit 13 may calculate, as an output, a value from 0 to 40 similarly to a PSS score which is the second data mentioned above, using the first stress calculation model.

Every time the first calculating unit 13 calculates the first stress value, the second calculating unit 14 calculates the second stress value on the basis of the first stress value, and the second data based on stress-related subjective data about the person U acquired from the person U in advance. Specifically, the second calculating unit 14 reads out the second stress calculation model stored on the second model storage unit 18, inputs the calculated first stress value, and the second data acquired in advance to the second stress calculation model, and thereby calculates the second stress value which is an output therefrom. That is, the second calculating unit 14 calculates the second stress value at preset timings such as at certain time intervals at which biometric data is acquired from the person U as mentioned above.

The output unit 15 outputs information based on the second stress value calculated at the second calculating unit 14. For example, every time the second stress value is calculated, in a case where the second stress value exceeds a preset criterion value, on the basis of which it is determined whether stress is high, the output unit 15 outputs an instruction to cause a display device 30 of an information processing device operated by an administrator at the workplace of the person U, a family member of the person U, or the like to display information to that effect (alert). Alternatively, every time the second stress value is calculated, the output unit 15 may always output an instruction such that the second stress value itself, that is, time-series changes of the second stress value of the person U, is displayed, or may output any data based on the second stress value. In addition, the output unit 15 may output data based on the second stress value to any person such as the target person U.

### Operation

Next, operations performed by the stress estimating device 10 mentioned above are explained mainly with reference to flowcharts in FIG. 3 to FIG. 4. First, an operation to be performed when the stress calculation model is generated by machine learning is explained with reference to the flowchart in FIG. 3.

First, as learning data, the stress estimating device 10 acquires the second data which is data based on stress-related subjective data about a certain plurality of people, here “PSS scores” obtained by totaling “perceived stress scales (Perceived Stress Scales (PSSs))” (step S1). As an example, the stress estimating device 10 acquires, as the second data, values from 0 to 40 obtained by totaling scores from 0 to 4 corresponding to five levels of answers to 10 question items like the ones depicted in FIG. 2 as the PSS scores. It should be noted that the stress estimating device 10 may acquire data represented by any values as the second data which is data based on stress-related subjective data about the people.

Thereafter, as learning data, the stress estimating device 10 acquires the first data which is data based on biometric data about the certain plurality of people (step S2). As an example, the stress estimating device 10 acquires, as the biometric data, heart rates of the people U or eye opening degrees extracted from facial images of the people U, and acquires, as the first data, the biometric data itself or feature values of the biometric data. It should be noted that, as the first data, the stress estimating device 10 may acquire any biometric data about the people.

In addition, as learning data, the data acquiring unit 11 acquires the third data which is data representing the stress-related condition of the people U at the time of measurement for the biometric data as mentioned above (step S3). As an example, the stress estimating device 10 asks stress-related questions to the people U around the timings of measurement for the biometric data, acquires and totals answers from the people U, and thereby acquires the third data based on stress-related subjective data about the people U. That is, the third data represents values of stress actually put on the people at the timings of measurement for the biometric data. It should be noted that, as the third data, the stress estimating device 10 may acquire data representing stress on the people by any method.

Thereafter, the stress estimating device 10 performs machine learning using the learning data including the first data, the second data, and the third data acquired as mentioned above, and generates a stress calculation model for calculating a stress value representing the stress-related condition of a person U (step S4). Specifically, first, the stress estimating device 10 calculates the first stress values from the biometric data itself about the people U obtained by measurement or the first data which is feature values of the biometric data. At this time, the stress estimating device 10 inputs the first data to the first stress calculation model stored on the first model storage unit 17, and thereby calculates the first stress values which are outputs therefrom. Note that the first stress calculation model is a model that is preset to calculate a stress value corresponding to biometric data about a person U. As an example, the stress estimating device 10 calculates, as the first stress value, a value from 0 to 40 similarly to a PSS score mentioned above by inputting, to the first stress calculation model, the first data which is biometric data about a person U. It should be noted that the stress estimating device 10 may calculate the first stress value in any format by inputting the first data to the first stress calculation model.

Next, the stress estimating device 10 generates the second stress calculation model that calculates the second stress value by performing learning using the calculated first stress values, and the second data which is stress-related subjective data like PSS scores acquired in advance from the people U. As an example, the stress estimating device 10 generates the second stress calculation model by performing machine learning using, as explanatory variables, the first stress values and second data described above, and using, as a response variable, the third data representing the stress-related condition of the people U at the time of acquisition of the biometric data from which the first stress values have been calculated. Then, the stress estimating device 10 stores the generated second stress calculation model on the second model storage unit 18.

Next, an operation of estimating stress on a target person using the generated stress calculation model is explained with reference to the flowchart in FIG. 4.

First, as the second data which is data based on stress-related subjective data about the target person U, the stress estimating device 10 acquires and stores a “PSS score” obtained by totaling “perceived stress scales (Perceived Stress Scales (PSSs))” in advance (step S11). The PSS score as the second data acquired at this time is data acquired similarly to the learning data mentioned above, and, as an example, the stress estimating device 10 acquires a value from 0 to 40 as the second data. It should be noted that, as data used for estimating stress, the stress estimating device 10 may acquire data represented by any values.

Next, the stress estimating device 10 acquires the first data which is data based on biometric data from the target person U at the timing of estimation of stress on the target person U (step S12). As an example, the stress estimating device 10 acquires, as the first data, biometric data itself like a heart rate of the person U or an eye opening degree extracted from a facial image of the person U, or feature values of the biometric data (step S13). Note that, as depicted as step S16, the stress estimating device 10 acquires the first data based on biometric data from the person U at preset timings, for example at certain time intervals, until the period of estimation of stress on the person ends. As an example, the stress estimating device 10 acquires the first data every day in a case where chronic stress on the person U is estimated, or every hour in a case where acute stress is measured. Note that the stress estimating device 10 may acquire any biometric data as the biometric data about the person.

Next, after acquiring the first data based on the biometric data from the person U, the stress estimating devices 10 calculates the first stress value by inputting the first data to the first stress calculation model (step S13). As an example, as the first stress value, the stress estimating device 10 calculates a value from 0 to 40 similarly to a PSS score which is the second data mentioned above.

Then, the stress estimating device 10 calculates the second stress value by inputting, to the second stress calculation model, the calculated first stress value, and the second data based on stress-related subjective data about the person U acquired from the target person U and stored in advance (step S14). Thereafter, the stress estimating device 10 outputs stress information about the target person U such as the calculated second stress value itself or an alert based on the second stress value (step S15).

Then, the stress estimating device 10 repeatedly performs the calculation of the second stress value similarly to the manner mentioned above until the stress estimation period of the target person U ends (No at step S16). That is, at the next timing of the stress estimation, the stress estimating device 10 repeats: the acquisition of the first data based on biometric data from the person U (step S12); the calculation of the first stress value using the first stress calculation model from the first data (step S13); the calculation of the second stress value by inputting, to the second stress calculation model, the calculated first stress value, and the second data based on stress-related subjective data about the person U acquired in advance (step S14). In this manner, the stress estimating device 10 keeps estimating the stress-related condition of the target person U.

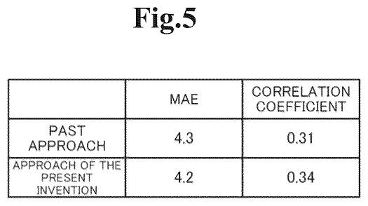

Here, advantageous effects achieved when stress values at two levels (the first stress value, the second stress value) are calculated using biometric data and stress-related subjective data as mentioned above are explained. An “MAE (Mean Absolute Error): mean absolute error” in FIG. 5 represents the average of the absolute values of differences between estimated values and correct answer values of stress values. As represented by the numerical values in this figure, the approach of the present invention is evaluated as giving a value smaller than that given by a past approach, and it can be known that the approach of the present invention achieves better precision. In addition, a “correlation coefficient” in FIG. 5 represents the magnitude and directionality of a relationship between estimated values and correct answer values of stress. As represented by the numerical values in this figure, the approach of the present invention is evaluated as giving a positive value greater than that given by the past approach, and it can be known that the approach of the present invention achieves better precision.

### Second Exemplary Embodiment

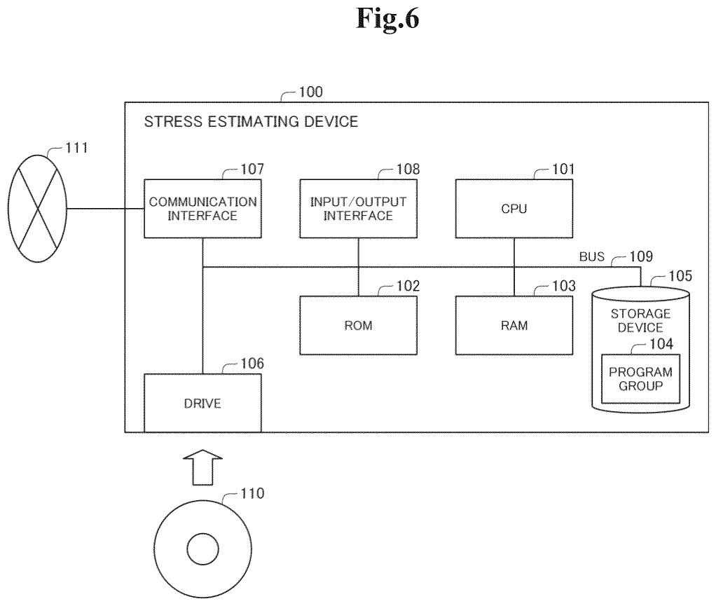

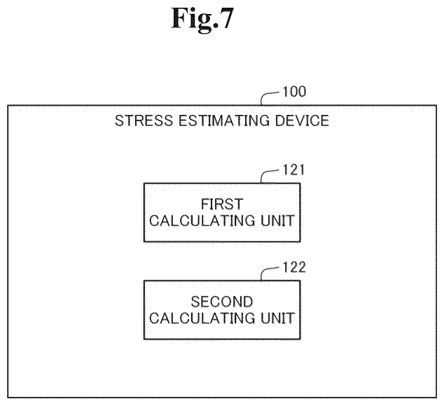

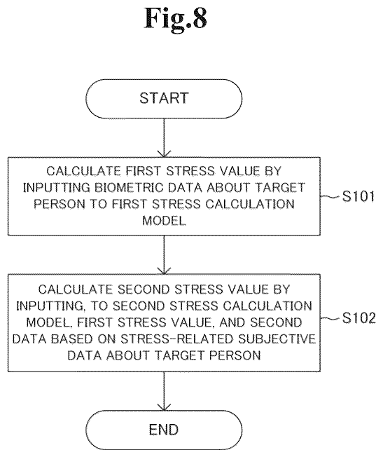

Next, a second exemplary embodiment of the present invention is explained with reference to FIG. 6 to FIG. 8. FIG. 6 to FIG. 7 are block diagrams depicting the configuration of a stress estimating device in the second exemplary embodiment, and FIG. 8 is a flowchart depicting an operation performed by the stress estimating device. Note that the present embodiment illustrates outlines of the configurations of the stress estimating device and the stress estimation method explained in the exemplary embodiment mentioned above.

First, the hardware configuration of a stress estimating device 100 in the present embodiment is explained with reference to FIG. 6. The stress estimating device 100 is configured using a typical information processing device, and has a hardware configuration as described below as an example.
Central Processing Unit (CPU) 101 (arithmetic device)
Read Only Memory (ROM) 102 (storage device)
Random Access Memory (RAM) 103 (storage device)
Program group 104 to be loaded to RAM 103
Storage device 105 having stored thereon the program group 104
Drive 106 that performs reading and writing on a storage medium 110 outside the information processing device
Communication interface 107 connected to a communication network 111 outside the information processing device
Input/output interface 108 for performing input/output of data
Bus 109 connecting the respective constituent elements

Then, the stress estimating device 100 can construct and have a first calculating unit 121 and a second calculating unit 122 depicted in FIG. 7 through acquisition of the program group 104 and execution thereof by the CPU 101. Note that the program group 104 is stored on, for example, the storage device 105 or the ROM 102 in advance, is loaded to the RAM 103 by the CPU 101, and is executed by the CPU 101 as needed. In addition, the program group 104 may be supplied to the CPU 101 via the communication network 111, or may be stored on the storage medium 110 in advance, read out by the drive 106, and supplied to the CPU 101. It should be noted that the first calculating unit 121 and the second calculating unit 122 mentioned above may be constructed using electronic circuits dedicated for realizing the means.

Note that FIG. 6 depicts an example of the hardware configuration of the information processing device that is the stress estimating device 100. The hardware configuration of the information processing device is not limited to that mentioned above. For example, the information processing device may be configured using part of the configuration mentioned above, such as without the drive 106.

Then, by functions of the first calculating unit 121 and the second calculating unit 122 constructed by programs as mentioned above, the stress estimating device 100 executes the stress estimation method depicted in the flowchart in FIG. 8.

As depicted in FIG. 8, the stress estimating device 100 is configured to execute processes of:
calculating the first stress value by inputting, to the first stress calculation model, the first data based on biometric data obtained by measurement from a target person (step S101); and
calculating the second stress value by inputting, to the second stress calculation model, the first stress value, and the second data based on stress-related subjective data acquired from the target person (step S102), in which
the second stress calculation model is generated in advance by learning using the first stress values and the second data about predetermined people acquired in the past.

By being configured in the manners described above, the present invention makes it possible to estimate stress on a person highly precisely by calculating stress values at two levels (the first stress value, the second stress value) using biometric data about the person and stress-related subjective data.

Note that the programs mentioned above can be supplied to a computer by being stored on a non-transitory computer readable medium of any type. Non-transitory computer readable media include tangible storage media of various types. Examples of non-transitory computer readable media include a magnetic recording medium (e.g. flexible disc, magnetic tape, hard disk drive), a magneto-optical recording medium (e.g. magneto-optical disc), a CD-ROM (Read Only Memory), a CD-R, a CD-R/W, and a semiconductor memory (e.g. mask ROM, PROM (Programmable ROM), EPROM (Erasable PROM), flash ROM, and RAM (Random Access Memory)). In addition, the programs may also be supplied to a computer by being stored on a transitory computer readable medium of any type. Examples of transitory computer readable media include electric signals, optical signals, and electromagnetic waves. A transitory computer readable medium can supply programs to a computer via a wired communication channel such as an electric wire or an optical fiber, or a wireless communication channel.

While the present invention has been explained thus far with reference to the exemplary embodiments and the like described above, the present invention is not limited to the exemplary embodiments mentioned above. The configurations and details of the present invention can be changed within the scope of the present invention in various manners that can be understood by those skilled in the art. In addition, at least one or more functions of the functions of the first calculating unit 121 and the second calculating unit 122 mentioned above may be executed at an information processing device installed and connected at any location on a network, that is, may be executed by so-called cloud computing.

### Supplementary Notes

Part of or the whole of the exemplary embodiments described above can also be described as in the following supplementary notes. Hereinbelow, outlines of the configurations of a stress estimation method, a stress estimating device, and a program in the present invention are explained. It should be noted that the present invention is not limited to the following configurations.

### Supplementary Note 1

A stress estimation method comprising:
calculating a first stress value by inputting, to a first stress calculation model, first data based on biometric data obtained by measurement from a target person; and
calculating a second stress value by inputting, to a second stress calculation model, the first stress value, and second data based on stress-related subjective data acquired from the target person, wherein
the second stress calculation model is generated in advance by learning using the first stress value and the second data about a predetermined person acquired in a past.

### Supplementary Note 2

The stress estimation method according to supplementary note 1, wherein the second stress calculation model is generated in advance by learning using, as explanatory variables, the first stress value calculated on a basis of biometric data obtained by measurement from the predetermined person, and the second data acquired in advance from the predetermined person, and using, as a response variable, third data related to stress acquired when the biometric data is obtained by measurement from the predetermined person.

### Supplementary Note 3

The stress estimation method according to supplementary note 2, wherein the third data is data based on stress-related subjective data about the predetermined person.

### Supplementary Note 4

The stress estimation method according to any one of supplementary notes 1 to 3, wherein
the stress estimation method comprises acquiring the second data from the target person in advance,
the first stress value is calculated by inputting, to the first stress calculation model, the first data based on the biometric data acquired from the target person thereafter, and
the second stress value is calculated by inputting, to the second stress calculation model, the calculated first stress value and the second data acquired in advance.

### Supplementary Note 5

The stress estimation method according to supplementary note 4, wherein
every time the biometric data is acquired from the target person, the first stress value is calculated by inputting, to the first stress calculation model, the first data based on the biometric data, and
every time the first stress value is calculated, the second stress value is calculated by inputting, to the second stress calculation model, the calculated first stress value and the second data acquired in advance.

### Supplementary Note 6

The stress estimation method according to supplementary note 4 or 5, wherein
at preset timings, the acquisition of the biometric data from the target person, and the calculation of the first stress value by inputting, to the first stress calculation model, the first data based on the acquired biometric data are repeatedly performed, and
every time the first stress value is calculated, the second stress value is calculated by inputting, to the second stress calculation model, the calculated first stress value and the second data acquired in advance, and information based on the calculated second stress value is output.

### Supplementary Note 7

The stress estimation method according to any one of supplementary notes 4 to 6, wherein the second data calculated on a basis of an answer from the target person to a preset question is acquired.

### Supplementary Note 8

A stress estimating device comprising:
a first calculating unit that calculates a first stress value by inputting, to a first stress calculation model, first data based on biometric data obtained by measurement from a target person; and
a second calculating unit that calculates a second stress value by inputting, to a second stress calculation model, the first stress value, and second data based on stress-related subjective data acquired from the target person, wherein
the second stress calculation model is generated in advance by learning using the first stress value and the second data about a predetermined person acquired in a past.

### Supplementary Note 9

The stress estimating device according to supplementary note 8, wherein the second stress calculation model is generated in advance by learning using, as explanatory variables, the first stress value calculated on a basis of biometric data obtained by measurement from the predetermined person, and the second data acquired in advance from the predetermined person, and using, as a response variable, third data related to stress acquired when the biometric data is obtained by measurement from the predetermined person.

### Supplementary Note 10

The stress estimating device according to supplementary note 9, wherein the third data is data based on stress-related subjective data about the predetermined person.

### Supplementary Note 11

The stress estimating device according to any one of supplementary notes 8 to 10, wherein
the stress estimating device comprises an acquiring unit that acquires the second data from the target person in advance,
the first calculating unit calculates the first stress value by inputting, to the first stress calculation model, the first data based on the biometric data acquired from the target person thereafter, and
the second calculating unit calculates the second stress value by inputting, to the second stress calculation model, the calculated first stress value and the second data acquired in advance.

### Supplementary Note 12

The stress estimating device according to supplementary note 11, wherein
every time the biometric data is acquired from the target person, the first calculating unit calculates the first stress value by inputting, to the first stress calculation model, the first data based on the biometric data, and
every time the first stress value is calculated, the second calculating unit calculates the second stress value by inputting, to the second stress calculation model, the calculated first stress value and the second data acquired in advance.

### Supplementary Note 13

The stress estimating device according to supplementary note 11 or 12, wherein
at preset timings, the first calculating unit repeatedly performs the acquisition of the biometric data from the target person, and the calculation of the first stress value by inputting, to the first stress calculation model, the first data based on the acquired biometric data, and
every time the first stress value is calculated, the second calculating unit calculates the second stress value by inputting, to the second stress calculation model, the calculated first stress value and the second data acquired in advance, and outputs information based on the calculated second stress value.

### Supplementary Note 14

The stress estimating device according to any one of supplementary notes 11 to 13, wherein the acquiring unit acquires the second data calculated on a basis of an answer from the target person to a preset question.

### Supplementary Note 15

A computer readable storage medium having stored thereon a program for causing an information processing device to execute processes of:
calculating a first stress value by inputting, to a first stress calculation model, first data based on biometric data obtained by measurement from a target person; and
calculating a second stress value by inputting, to a second stress calculation model, the first stress value, and second data based on stress-related subjective data acquired from the target person, wherein
the second stress calculation model is generated in advance by learning using the first stress value and the second data about a predetermined person acquired in a past.

### REFERENCE SIGNS LIST

10 stress estimating device
11 data acquiring unit
12 learning unit
13 first calculating unit
14 second calculating unit
15 output unit
16 person information storage unit
17 first model storage unit
18 second model storage unit
20 input device
30 display device
100 stress estimating device
101 CPU
102 ROM
103 RAM
104 program group
105 storage device
106 drive
107 communication interface
108 input/output interface
109 bus
110 storage medium
111 communication network
121 first calculating unit
122 second calculating unit

What is claimed is:

1. A stress estimation method comprising:
calculating a first stress value by inputting, to a first stress calculation model, first data based on biometric data obtained by measurement from a target person; and
calculating a second stress value by inputting, to a second stress calculation model, the first stress value, and second data based on stress-related subjective data acquired from the target person, wherein
the second stress calculation model is generated in advance by learning using the first stress value and the second data about a predetermined person acquired in a past.

2. The stress estimation method according to claim 1, wherein the second stress calculation model is generated in advance by learning using, as explanatory variables, the first stress value calculated on a basis of biometric data obtained by measurement from the predetermined person, and the second data acquired in advance from the predetermined person, and using, as a response variable, third data related to stress acquired when the biometric data is obtained by measurement from the predetermined person.

3. The stress estimation method according to claim 2, wherein the third data is data based on stress-related subjective data about the predetermined person.

4. The stress estimation method according to claim 1, wherein
the stress estimation method comprises acquiring the second data from the target person in advance,
the first stress value is calculated by inputting, to the first stress calculation model, the first data based on the biometric data acquired from the target person thereafter, and
the second stress value is calculated by inputting, to the second stress calculation model, the calculated first stress value and the second data acquired in advance.

5. The stress estimation method according to claim 4, wherein
every time the biometric data is acquired from the target person, the first stress value is calculated by inputting, to the first stress calculation model, the first data based on the biometric data, and
every time the first stress value is calculated, the second stress value is calculated by inputting, to the second stress calculation model, the calculated first stress value and the second data acquired in advance.

6. The stress estimation method according to claim 4, wherein
at preset timings, the acquisition of the biometric data from the target person, and the calculation of the first stress value by inputting, to the first stress calculation model, the first data based on the acquired biometric data are repeatedly performed, and
every time the first stress value is calculated, the second stress value is calculated by inputting, to the second stress calculation model, the calculated first stress value and the second data acquired in advance, and information based on the calculated second stress value is output.

7. The stress estimation method according to claim 4, wherein the second data calculated on a basis of an answer from the target person to a preset question is acquired.

8. A stress estimating device comprising:
at least one memory configured to store instructions; and
at least one processor configured to execute instructions to:
calculate a first stress value by inputting, to a first stress calculation model, first data based on biometric data obtained by measurement from a target person; and
calculate a second stress value by inputting, to a second stress calculation model, the first stress value, and second data based on stress-related subjective data acquired from the target person, wherein
the second stress calculation model is generated in advance by learning using the first stress value and the second data about a predetermined person acquired in a past.

9. The stress estimating device according to claim 8, wherein the second stress calculation model is generated in advance by learning using, as explanatory variables, the first stress value calculated on a basis of biometric data obtained by measurement from the predetermined person, and the second data acquired in advance from the predetermined person, and using, as a response variable, third data related to stress acquired when the biometric data is obtained by measurement from the predetermined person.

10. The stress estimating device according to claim 9, wherein the third data is data based on stress-related subjective data about the predetermined person.

11. The stress estimating device according to claim 8, wherein
the at least one processor is configured to execute the instructions to:
acquire the second data from the target person in advance,
calculate the first stress value by inputting, to the first stress calculation model, the first data based on the biometric data acquired from the target person thereafter, and
calculate the second stress value by inputting, to the second stress calculation model, the calculated first stress value and the second data acquired in advance.

12. The stress estimating device according to claim 11, wherein
the at least one processor is configured to execute the instructions to:
every time the biometric data is acquired from the target person, calculate the first stress value by inputting, to the first stress calculation model, the first data based on the biometric data, and
every time the first stress value is calculated, calculate the second stress value by inputting, to the second stress calculation model, the calculated first stress value and the second data acquired in advance.

13. The stress estimating device according to claim 11, wherein
the at least one processor is configured to execute the instructions to:
at preset timings, repeatedly perform the acquisition of the biometric data from the target person, and the calculation of the first stress value by inputting, to the first stress calculation model, the first data based on the acquired biometric data, and
every time the first stress value is calculated, calculate the second stress value by inputting, to the second stress calculation model, the calculated first stress value and the second data acquired in advance, and output information based on the calculated second stress value.

14. The stress estimating device according to claim 11, wherein the at least one processor is configured to execute the instructions to acquire the second data calculated on a basis of an answer from the target person to a preset question.

15. A non-transitory computer readable storage medium having stored thereon a program comprising instructions for causing an information processing device to execute processes of:
calculating a first stress value by inputting, to a first stress calculation model, first data based on biometric data obtained by measurement from a target person; and
calculating a second stress value by inputting, to a second stress calculation model, the first stress value, and second data based on stress-related subjective data acquired from the target person, wherein
the second stress calculation model is generated in advance by learning using the first stress value and the second data about a predetermined person acquired in a past.
